(19) United States
(12) Patent Application Publication
(10) Pub. No.: US 2024/0117316 A1
(43) Pub. Date: Apr. 11, 2024

(54) METHOD FOR PREPARING MESENCHYMAL STEM CELL-DERIVED EXTRACELLULAR VESICLE, MESENCHYMAL STEM CELL-DERIVED EXTRACELLULAR VESICLE PREPARED BY THE METHOD, AND USE OF MESENCHYMAL STEM CELL-DERIVED EXTRACELLULAR VESICLE FOR REDUCING ADIPOGENESIS AND TREATING OSTEOARTHRITIS

(71) Applicant: GWO XI STEM CELL APPLIED TECHNOLOGY CO., LTD., Hsinchu County (TW)

(72) Inventors: Yu-Chen Tsai, Hsinchu County (TW); Ming-Hsi Chuang, Hsinchu County (TW); Po-Cheng Lin, Hsinchu County (TW)

(21) Appl. No.: 18/376,566

(22) Filed: Oct. 4, 2023

Related U.S. Application Data
(60) Provisional application No. 63/414,916, filed on Oct. 11, 2022.

(30) Foreign Application Priority Data
Sep. 27, 2023 (TW) 112137035

(51) Int. Cl. C12N 5/0775; A61K 35/28

(52) U.S. Cl. CPC C12N 5/0665; A61K 35/28

(57) ABSTRACT

The present disclosure provides a method for preparing mesenchymal stem cell-derived extracellular vesicle, the mesenchymal stem cell-derived extracellular vesicle prepared by the method, and use of the mesenchymal stem cell-derived extracellular vesicle for reducing adipogenesis and treating osteoarthritis. The mesenchymal stem cell-derived extracellular vesicle of the present disclosure achieves the effect of reducing adipogenesis and treating osteoarthritis through various efficacy experiments.

### STATEMENT REGARDING SEQUENCE LISTING

The sequence listing associated with this application is provided in text format in lieu of a paper copy and is hereby incorporated by reference into the specification. The name of the XML file containing the sequence listing is 112F0464-IE_Sequence_listing. The XML file is 9206 bytes; was created on Oct. 4, 2023.

### CROSS-REFERENCE TO RELATED APPLICATION

This application claims priorities of Provisional application No. 63/414,916, filed on Oct. 11, 2022, and Taiwan patent application No. 112137035, filed on Sep. 27, 2023 the content of which is incorporated herein in its entirety by reference.

### BACKGROUND OF THE INVENTION

### 1. Field of the Invention

The present invention relates to a method for preparing mesenchymal stem cell-derived extracellular vesicle, the mesenchymal stem cell-derived extracellular vesicle prepared by the method, and use of the mesenchymal stem cell-derived extracellular vesicle for reducing adipogenesis and treating osteoarthritis.

### 2. The Prior Art

The cause of obesity is mainly due to the imbalance of energy metabolism in the human body, resulting in an increase in fat cells. Since cells mainly use glucose as the form of energy intake, when the energy intake is greater than the energy consumed, in order to store the excess energy, in addition to converting the excess glucose into glycogen for storage, cells also convert part of the glucose into triglycerides. Triglycerides are stored in adipose tissue, so at the same time, excess energy would also promote the differentiation of adipocyte precursor cells to form adipocytes, thereby increasing the accumulation of adipocytes and the formation of adipose tissue, leading to obesity. On the other hand, obesity can also lead to other diseases, such as arthritis.

Arthritis is one of the most common chronic diseases in the world. It is mainly caused by the deterioration of the cartilage of the joints or inflammation of the connective tissue, which causes joint pain and interferes with the normal movement of the joints. There are more than one hundred types in total. There are many causes of arthritis, and joint damage is also different. There are approximately 355 μmillion arthritis patients in the world, of which more than 100 μmillion are in China. In the United States, one in five people suffers from arthritis pain and discomfort; in Asia, one in six people suffers from the disease at some point in their lives. Arthritis is not a disease unique to the elderly, it can affect all ages, including children.

Arthritis can occur in the back, neck, knees, shoulder joints, hands, hip joints, and ankles. Most arthritis is related to human aging. People over sixty years old would suffer from some types of arthritis but young people would also suffer from arthritis. There are more than one hundred types of osteoarthritis (OA), rheumatoid, rheumatic and septic arthritis, traumatic osteoarthritis, and autoimmune arthritis, and ankylosing arthritis is also a type of arthritis. In particular, osteoarthritis mainly affects articular cartilage and mainly occurs in movable joints such as knees, hips, cervical vertebrae, and finger joints. Osteoarthritis often causes joint swelling, pain, stiffness, immobility, and loss of function. Since articular cartilage lacks a self-repair mechanism, once damaged, it often deteriorates into degenerative arthritis. In current society, the population is getting older and the number of osteoarthritis cases is increasing exponentially. Providing effective treatments for osteoarthritis is a very important and difficult issue. In recent years, the age of onset of osteoarthritis has been on a downward trend, and it mostly occurs in younger patients. In most cases, the main causes of osteoarthritis in young people are obesity and excessive exercise. Therefore, how to generate chondrocytes and reduce adipogenesis has become an important issue in the treatment of osteoarthritis.

At present, clinical drug treatments for reducing adipogenesis and osteoarthritis have limited effect and have serious side effects, and many patients cannot continue to treat. More importantly, the drug only alleviates the symptoms, but fails to fundamentally solve the problem, so how to develop a new drug that can effectively reduce adipogenesis and treat osteoarthritis is an important issue that the present invention intends to solve here.

In order to solve the above-mentioned problems, those skilled in the art urgently need to develop a novel pharmaceutical composition for reducing adipogenesis and treating osteoarthritis for the benefit of a large group of people in need thereof.

### SUMMARY OF THE INVENTION

A primary objective of the present invention is to provide a method for preparing a mesenchymal stem cell (MSC)-derived extracellular vesicle (EV), comprising the following steps: (a) providing a human mesenchymal stem cell; (b) culturing and maintaining the human mesenchymal stem cell in a keratinocyte serum-free medium (KSFM) supplemented with fetal bovine serum (FBS), N-acetyl-L-cysteine and L-ascorbic acid 2-phosphate; (c) collecting a cultured human mesenchymal stem cell, and removing cell debris using a centrifugation process; and (d) filtering supernatant thus formed to obtain the mesenchymal stem cell (MSC)-derived extracellular vesicle (EV).

Another objective of the present invention is to provide a mesenchymal stem cell (MSC)-derived extracellular vesicle (EV), which is prepared by the aforementioned method.

Another objective of the present invention is to provide a method for reducing adipogenesis, comprising administering to a subject in need thereof a pharmaceutical composition comprising an effective amount of the aforementioned mesenchymal stem cell (MSC)-derived extracellular vesicle (EV).

Another objective of the present invention is to provide a method for treating osteoarthritis (OA), comprising administering to a subject in need thereof a pharmaceutical composition comprising an effective amount of the aforementioned mesenchymal stem cell (MSC)-derived extracellular vesicle (EV).

According to an embodiment of the present invention, the human mesenchymal stem cell is adipose-derived mesenchymal stem cell (ADSC).

According to an embodiment of the present invention, the mesenchymal stem cell-derived extracellular vesicle has a size of 30 nm-1 μm.

According to an embodiment of the present invention, the mesenchymal stem cell-derived extracellular vesicle comprises a cartilage-related gene selected from the group consisting of: interleukin-2 (IL-2), Uteroglobin, alpha-fetoprotein (AFP), angiopoietin-like 6 (ANGPTL-6), fatty acid-binding protein-1 (FABP-1), cartilage oligomeric matrix protein (COMP), platelet-derived growth factor AA (PDGF-AA), granulocyte-colony stimulating factor (G-CSF), and a combination thereof.

According to an embodiment of the present invention, the mesenchymal stem cell (MSC)-derived extracellular vesicle (EV) is adipose-derived mesenchymal stem cell (ADSC)-derived extracellular vesicle (EV).

According to an embodiment of the present invention, the mesenchymal stem cell-derived extracellular vesicle upregulates type II collagen.

According to an embodiment of the present invention, the mesenchymal stem cell-derived extracellular vesicle decreases lipid accumulation rate during adipogenic induction.

According to an embodiment of the present invention, the mesenchymal stem cell-derived extracellular vesicle reduces lipid droplet formation.

According to an embodiment of the present invention, the mesenchymal stem cell-derived extracellular vesicle produces chondrocyte.

According to an embodiment of the present invention, the osteoarthritis is caused by obesity.

According to an embodiment of the present invention, the pharmaceutical composition is in a dosage form for parenteral administration.

In summary, the mesenchymal stem cell (MSC)-derived extracellular vesicle (EV) of the present invention achieves the effect on reducing adipogenesis and treating osteoarthritis through upregulating type II collagen, decreaseing lipid accumulation rate during adipogenic induction, and reducing lipid droplet formation.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIGS. 1A-1C show effects of adipose-derived stem cells (ADSC) differentiation on specific lineages in differentiation medium with EV-depleted FBS. ADSCs from passage 5 were cultured in various differentiation induction media with 10% FBS. (FIG. 1A) ADSCs were cultured in adipogenic differentiation medium with EV-depleted FBS for 14 days and observed by microscopy, followed by Oil Red O staining. (FIG. 1B) Cells were cultured in osteogenic differentiation medium with EV-depleted FBS for 14 days and observed by microscopy, followed by alkaline phosphatase (ALP) staining. (FIG. 1C) For chondrogenic induction, cells were maintained in differentiation medium and observed by microscopy, followed by Alcian blue staining on day 14. The histology of paraffin-embedded tissue sections was evaluated on day 21 of chondrogenic differentiation. The differentiation quantifications of tissues were performed after 14 days of adipogenic, osteogenic differentiation, and after 21 days of chondrogenic differentiation, respectively. The percentage positive area was calculated using ImageJ software. The mean±standard deviation (SD) levels of tissue differentiation in the three groups. *p<0.05; **p<0.01; ***p<0.001, when EV-depleted FBS group was compared to the control group.

FIG. 2 shows adipogenic capacity of mesenchymal stem cells (MSCs) from different tissue sources. Two different tissue-derived mesenchymal stem cells, ADSCs and Wharton's jelly (WJ)-MSCs, were cultured in adipogenic differentiation medium for 14 days, and then the Western blot method was used to observe the amount of peroxisome proliferator activated receptor γ (PPARγ) protein in ADSCs and WJ-MSCs. Further ImageJ analyses of the bands were performed to measure the protein expression levels. The ratios of PPARγ protein expression was normalized to glyceraldehyde-3-phosphate dehydrogenase (GAPDH).

FIGS. 3A-3C show effects of chondrogenic differentiation capacity by secreted substances of ADSCs. (FIG. 3A) Scheme of the experimental procedure. ADSCs were seeded at low density (5×10³ cells/well) in the lower well, and cells at high cell densities (8×10⁴ ADSCs/well) were seeded in the upper chamber of 8.0-μm Transwell plates. The control group was the upper chamber without seeding cells. (FIG. 3B) The images recorded were used in the coculture system. Cartilage tissue was collected from the co-culture group. (FIG. 3C) The sliced cartilage tissue was stained with Alcian blue, and then cartilage differentiation status was obtained by microscopy. The nucleus was stained red-purple, and the cytoplasm was stained light pink.

FIGS. 4A-4E show monitoring of mesenchymal stem cell (MSC) characteristics under serum-free culture conditions. The control group was cultured in medium with 10% FBS, and the serum-free group was serum starved of ADSCs for 24 h before differentiation. (FIG. 4A) ADSC morphology was observed by microscopy. Scale bar, 50 μm. (FIG. 4B) Flow cytometry was used to determine surface marker expression, including CD14, CD19, CD34, CD45, HLA-DR, CD73b, CD90, and CD105. ADSC differentiation was induced in different types of differentiation media at passage 5. (FIG. 4C) Cells that had undergone adipogenic differentiation were fixed in 4% formaldehyde, and Oil Red O staining of ADSCs for adipogenic induction was observed by microscopy at 7, 14, and 21 days. (FIG. 4D) Cells were subsequently cultured in osteogenic differentiation medium for 7, 14, and 21 days and observed by microscopy, followed by ALP staining. (FIG. 4E) For chondrogenic induction, cells were maintained in differentiation medium for 21 days. Tissue sections were stained with Alcian blue and evaluated by microscopy. The mean±standard deviation (SD) levels of chondrogenic differentiation in the three groups. A p value higher than 0.05 is denoted by “ns” (non-significant).

FIGS. 5A-5D show extraction and identification of MSC-derived EVs. (FIG. 5A) Using nanoparticle tracking analysis (NTA), the EV production yield from ADSCs and WJ-MSCs was analyzed by the average particle numbers in each cell. (FIG. 5B) The average size of EVs was determined by NTA. (FIG. 5C) The protein levels of EV markers, including Alix, TSG101, CD9, CD81, and HSP70, were analyzed from EV samples using Western blot analysis. Analysis of Calnexin, which is not expressed by EV, in ADSC lysates and EV samples using Western blotting. (FIG. 5D) The stock solution FBS was diluted with DPBS to a final concentration of 10% FBS. The particle concentration of EVs from 10% FBS was analyzed by NTA. This was the analysis result of the three samples. That is, the results of 3 separate samplings, each sample would be analyzed 3 times. The difference was considered statistically significant if p values were less than 0.05. P values less than 0.05 are denoted by by “*”, p values less than 0.01 are denoted by “**”, and p values higher than 0.05 are denoted by “ns” (non-significant).

FIGS. 6A and 6B show effects of EVs on stem cell differentiation into chondrocytes. Normal FBS was added to the culture medium used for the MSC differentiation process in the regular condition group (Normal). Cells from the EV-free control group were maintained in medium with 10% EV-depleted FBS during the differentiation process. Cells from the EV test groups were cultured in a medium containing EV-deleted FBS. Cells were added with EVs derived from ADSCs and WJ-MSCs in chondrogenic differentiation medium. The EV concentrations were 5×10⁷ and 5×10⁸, respectively. The medium was completely replaced every 3 days. (FIG. 6A) Cells were cultured in chondrogenic differentiation medium for 21 days. Tissue sections were stained and observed by microscopy, followed by Alcian blue staining. (FIG. 6B) On day 14, cells were harvested, and lysis buffer was used to extract the proteins. Type II collagen and cyclins (A2, B1, and D1) protein expression levels were assessed by Western blot analysis. Further ImageJ analyses of the bands were performed to measure the protein expression levels. GAPDH represents glyceraldehyde-3-phosphate dehydrogenase.

FIGS. 7A-7D show quantitative real-time gene expression analysis of chondrogenic-specific genes. Normal FBS was added to the culture medium used for the MSC differentiation process in the regular condition group (Normal). Cells from the EV-free control group were maintained in medium with 10% EV-depleted FBS during the differentiation process. Cells from the EV test groups were cultured in a medium containing EV-deleted FBS. Cells were added with EVs derived from ADSCs and WJ-MSCs in chondrogenic differentiation medium. The mRNA expression levels of (FIG. 7A) type I collagen (COLI), (FIG. 7B) COLII, (FIG. 7C) aggrecan (ACAN), and (FIG. 7D) SOX9, were measured in chondrogenic differentiated tissues after 21 days. GAPDH was used as an internal control. The value of fold change was normalized to the regular culture condition, and all values are expressed as mean±SEM, mL=3. *p<0.05; **p<0.01, compared to the EV-free group using t-test. A p value higher than 0.05 is denoted by “ns” (non significant).

FIGS. 8A-8C show effects of EVs on stem cell differentiation into adipocytes. During the differentiation process, EVs derived from ADSCs and WJ-MSCs were added to adipogenic differentiation medium and treated with different EV concentrations. The medium was replaced every 3 days. (FIG. 8A) On days 7 and 14, lipid accumulation during adipogenic induction was monitored by Oil Red O staining. Hematoxylin-stained cell nuclei were stained purplish blue. (FIG. 8B) Lipid droplets were dissolved in methanol, and the amount of oil droplet generation was quantified. (FIG. 8C) On day 14, cells were harvested, and lysis buffer was used to extract the proteins. AT-rich interactive domain 5A (Arid5a) and PPARγ protein expression levels were assessed by Western blot analysis. Further ImageJ analyses of the bands were performed to measure the protein expression levels. The ratios of Arid5a and PPARγ protein expression were normalized to GAPDH. *p<0.05; **p<0.01, compared to the normal group using t-test.

FIGS. 9A and 9B show the second donor's adipogenic differentiation and chondrogenic differentiation experiments. (FIG. 9A) On days 14, lipid accumulation during adipogenic induction was monitored by Oil Red O staining. Hematoxylin-stained cell nuclei were stained purplish blue. (FIG. 9B) Cells were cultured in chondrogenic differentiation medium for 21 days. Tissue sections were stained and observed by microscopy, followed by Alcian blue staining.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

According to the present invention, the term “adipose-derived stem cells (ADSCs)” refers to mesemchymal stem cells separated from fat, which are multipotent stem cells having high plasticity. After induction, they can be differentiated into cells of many different tissues.

According to the present invention, the extracellular vesicle comprises exosome and microvesicles.

As used herein, the term “treating” or “treatment” refers to alleviating, reducing, ameliorating, relieving, or controlling one or more clinical signs of a disease or disorder, and lowering, stopping, or reversing the progression of severity regarding the condition or symptom being treated.

According to the present invention, the pharmaceutical composition can be manufactured to a dosage form suitable for parenteral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, and intralesional injection.

The pharmaceutical composition according to the present invention can comprise a pharmaceutically acceptable carrier which is widely used in pharmaceutical manufacturing technology. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

The procedure of expansion of human mesenchymal stem cells (MSCs) is as follows. In this experiment, we used human MSCs from three healthy donors. Human adipose-derived mesenchymal stem cells (ADSCs) from two donors were tested for chondrogenic and adipogenic differentiation and one of ADSCs was used as a tool for EV production; Wharton's jelly (WJ)-MSCs (RM60596) were purchased from the Bioresource Collection and Research Centre (BCRC), Hsinchu, Taiwan, and were also used as an EV production tool. All cells were provided by Gwo Xi Stem Cell Applied Technology Co., Ltd. (Taipei City, Taiwan) and donors signed a consent form for cell donation.

The cells were cultured and maintained in keratinocyte serum-free medium (KSFM; Invitrogen-Gibco, Grand Island, NY, USA) supplemented with 10% (v/v) fetal bovine serum (FBS; Hyclone, Logan, UT, USA), antioxidants N-acetyl-L-cysteine (Sigma-Aldrich, St. Louis, MO, USA), and L-ascorbic acid 2-phosphate (Sigma-Aldrich). The cells were incubated at 37° C. with 5% CO₂ until the monolayer of adherent cells reached 70-80% confluence. During the experiment, the serum-free medium (SFM) was keratinocyte serum-free medium containing N-acetyl-L-cysteine and L-ascorbic acid 2-phosphate, which was used as a production medium for collecting EVs. Exosome-depleted FBS (EV-depleted FBS) was purchased from Gibco (Invitrogen-Gibco). The cells were used for the study in the passages 5.

The procedure of flow cytometry analysis is as follows. The ADSCs were seeded into a 175-cm² flask at a density of 5×10⁵ cells per flask. After growth for four days, culture medium was replaced with serum-free media for 24 h, and then cells were harvested. The surface phenotypes of ADSCs were characterized using a BD Accuri C6 flow cytometer (Becton Dickinson, Franklin Lakes, NJ, USA), and cells were stained with antibodies against the human clusters of differentiation (CD): CD14, CD19, CD34, CD45, CD73, CD90, CD105, and HLA-DR (Becton Dickinson). Labeling was performed according to the manufacturer's instructions.

The procedure of ADSC differentiation capability is as follows. ADSCs were seeded in 24-well plates at a density of 5000 cells/well in culture medium containing 10% FBS. After 24 h, the culture medium was removed, and cells were washed twice with Dulbecco's phosphate buffered saline (DPBS; Corning, Manassas, VA, USA) without calcium and magnesium. Cells were placed in SFM for 24 h. ADSCs were then induced to differentiate into adipogenic, osteogenic, and chondrogenic lineages using differentiation medium for 21 days.

The procedure of adipogenic differentiation is as follows. For adipogenic differentiation, ADSCs were incubated with PRIME-XV Adipogenic Differentiation SFM (Irvine Scientific, Santa Ana, CA, USA) containing 10% FBS, and the medium was exchanged every 3 days. On days 7, 14, and 21, the adipogenic differentiation of ADSCs was fixed in 4% formaldehyde solution (Sigma-Aldrich) for Oil Red O staining (ScyTek, BH Hague, Netherlands). Cells were observed under a light microscope.

The procedure of osteogenic differentiation is as follows. For osteogenic differentiation, PRIME-XV Osteogenic Differentiation SFM (Irvine Scientific) supplemented with 10% FBS was added to the wells for 21 days. On days 7, 14, and 21, the osteogenic differentiation of ADSCs was detected using an alkaline phosphatase (ALP) detection kit (Sigma-Aldrich) according to the manufacturer's instructions. Stained cells were captured as images under a light microscope, as described previously.

The procedure of chondrogenic differentiation is as follows. ADSCs were cultured in PRIME-XV Chondrogenic Differentiation SFM (Irvine Scientific) containing 10% FBS for 21 days. After chondrogenic induction, the spheroid formation of ADSCs exhibited cartilage-like features. Chondrogenic pellets were subjected to histological staining. The pellets were fixed in 4% formaldehyde, dehydrated using alcohol, cleared with xylene (EMD; J. T. Baker, CAS 1330-20-7), and embedded in paraffin. Finally, paraffin-embedded tissue was cut into 4-μm-thick sections, placed on glass slides, and dewaxed. Alcian blue staining (Scy-Tek) was used to determine the production of sulfated glycosaminoglycans (GAGs). Cells were observed under a light microscope and images were captured.

The procedure of co-culture system is as follows. ADSCs were seeded in 12-well plates at a density of 5000 cells/well and the upper chamber of 8.0-μm Transwell plates (Corning) at a high density of 8×10⁴ ADSCs. Cells were cultured overnight in a medium supplemented with 10% FBS and then rinsed twice with DPBS after removing the culture medium. The replacement culture medium of the upper chambers was culture medium containing 10% EV-depleted FBS, and the lower chambers were replaced with chondrogenic differentiation medium containing 10% EV-depleted FBS. The medium was refreshed every 3 days. On day 21, the efficiency of cartilage differentiation was assessed by histochemical staining (Alcian blue). Cells were analyzed under a light microscope (Olympus BX43, Tokyo, Japan). Commercially available EV-depleted FBS was purchased from Gibco (Life Technologies, Waltham, MA, USA).

Extracellular vesicles (EVs) are heterogeneous particles formed by outward budding or exocytosis of original cells, and have no functional nuclei and cannot be replicated. Extracellular vesicles include exosomes and microvesicles. The diameter of extracellular vesicles ranges from 30 nm to 1 μm, of which the diameter of exosomes ranges from 30 to 150 nm and the diameter of microvesicles ranges from 100 to 1000 nm. Extracellular vesicles are cell-derived particles wrapped in a lipid bilayer membrane and contain proteins, lipids, nucleic acids, etc. The procedure of isolation of EVs by ultrafiltation (UF) is as follows. MSCs were collected from SFM, and two centrifugation processes were used to remove cell debris. First, the medium was centrifuged at 300×g for 5 μmin at 4° C. After centrifugation, the supernatant was transferred to a new tube and then centrifuged at 4000×g for 20 μmin at 4° C. After performing the differential centrifugation steps, EVs were collected from the supernatant. The supernatant was then filtered through a 0.22 μm filter (Merck Millipore, Billerica, MA, USA) to remove large vesicles. Amicon Ultra-15 with a molecular weight exceeding 100 kDa (Millipore) was used to remove the free protein, followed by centrifugation at 4000×g. The final volume depended on the subsequent steps. For standard preparation, medium was further concentrated 25-fold. The particle numbers and size were measured using NTA. The EV sample was kept at −80° C. until use.

The procedure of nanoparticle tracking analysis (NTA) is as follows. In the present invention, we used nanoparticle tracking analysis (NTA, NanoSight NS300; Malvern Panalytical, Malvern, UK) for analysis of EV concentration and size distribution according to the manufacturer's instructions. The collected medium was centrifuged to remove cell debris. Next, we used production medium diluted with water at a concentration range of 1×10⁶-1×10⁹ particles per milliliter for the NTA assay. One mL of dilution sample was injected into the sample chamber with sterile syringes, and particle concentration and size of samples were analyzed under a constant flow rate of 70 μL per minute at room temperature.

The procedure of Western blot analysis is as follows. We used Western blot analysis to detect the expression of positive markers of exosomes, i.e., transmembrane proteins Alix, TSG101, heat shock protein 70 (HSP70), CD9, and CD81. Calnexin in the endoplasmic reticulum, a negative marker, was used as one of the indicators of EVs purity. Culture supernatants were concentrated and transferred to sterile tubes, and then samples were incubated with Exo-Quick-TC (System Biosciences, Palo Alto, CA, USA) at 4° C. for exosome precipitation. The exosome pellets were dissolved in the protein lysis buffer. After lysis, the samples were centrifuged to obtain the exosome proteins. Twenty-five g of each sample was loaded onto a 10% sodium dodecyl sulfate polyacrylamide gel (SDS-PAGE). After electrophoresis, proteins were transferred to a PVDF membrane, probed with primary antibody followed by incubation with horseradish peroxidase (HRP)-coupled secondary antibody against the primary antibody. After incubating with the HRP-coupled secondary antibodies, visualization of the protein bands was performed by incubating with chemiluminescent substrate (Thermo Fisher Scientific) and the results were documented. Alix and Calnexin were purchased from Cell Signaling Technology (Danvers, MA, USA); the antibody against Tumor susceptibility gene 101 (TSG101) was purchased from Santa Cruz Biotechnology (Dallas, Texas, USA); HSP70 was purchased from BD Biosciences (Franklin Lakes, NJ, USA); CD9 and CD81 were purchased from R&D Systems, Minneapolis, MN, USA.

The procedure regarding detection of MSC-derived functional EV via chondrogenic differentiation is as follows. During the chondrogenic differentiation process, ADSCs were cultured in EV-free FBS medium. EVs derived from ADSCs and WJ-MSCs were added to chondrogenic differentiation medium. The EV concentrations were 5×10⁷ and 5×10⁸, respectively. The medium was completely replaced every 3 days. At 14 days, cells were harvested, and the protein expression of COLII and cyclins (A2, B1, and D) (Cell Signaling Technology) was detected by chondrocyte induction. In addition, the protein expression levels were quantitated by ImageJ software. At 21 days, cartilage tissue was stained by Alcian blue, and the nuclei were stained with Nuclear Fast Red. Cells were observed under a light microscope and images were captured.

The procedure regarding detection of MSC-derived functional EV via lipid droplets formation is as follows. During the differentiation process, ADSCs were cultured in adipogenic differentiation medium containing 5×10⁷ and 5×10⁸ EVs. The medium was replaced every 3 days. EVs were released from ADSCs and WJ-MSCs. On days 7 and 14, adipose-differentiated cells were stained by Oil Red 0, and the accumulation of oil droplets was observed under a microscope. After imaging, oil red O-stained intercellular oil droplets were eluted with isopropanol and quantified by absorbance reading at 510 nm.

The procedure regarding RNA isolation and cDNA synthesis is as follows. Total RNA was extracted from cartilage tissue in EV-induced chondrogenic differentiation experiments using a GENEzol™ TriRNA Pure Kit (Geneaid) according to the manufacturer's instructions. RNA measurement was conducted by measuring absorbance at 260 nm and 280 nm using an Epoch™ Microplate Spectrophotometer (Bio-Tek). One 1 g of total RNA in a volume of 20 μl was used to synthesize single-stranded cDNA using a High Capacity cDNA Reverse Transcription Kit (Applied Biosystems, Carlsbad, CA, USA). The reaction conditions were incubation at 25° C. for 10 μmin, followed by 37° C. for 120 μmin and the reaction was terminated by heating at 85° C. for 5 μmin. Finally, the cDNA products were stored at −20° C. until use.

The procedure regarding quantitative real-time PCR is as follows. To examine the expression patterns of chondrogenic-related genes in the ADSCs, quantitative real-time PCR (qRT-PCR) was performed. The gene-specific primers were provided by the Department of Medical Research, Far Eastern Memorial Hospital, Taiwan. The primer sequences used for qRT-PCR are listed in Table 1, and GAPDH was used as an internal control for normalization of gene expression. The qRT-PCR was conducted with iTaq™ Universal SYBR® Green Supermix (Bio-Rad) on a realtime PCR System. The PCR temperature cycling conditions were as follows: initial denaturation for 10 μmin at 95° C., followed by 45 standard cycles: denaturation at 95° C. for 10 s, primer annealing for 10 s at 55° C., and primer extension at 72° C. for 10 s. All reactions were conducted in triplicate.

TABLE 1

| Gene | Primer | Sequence (5'-3') | Accession No. |
|---|---|---|---|
| Human | Forward | GTCAAGGCTGAGAACGGGAA (SEQ | NM_002046.7 |
| GAPDH | | ID NO: 1) | |
| | Reverse | GCAGAGGGGGCAGAGATGAT | |
| | | (SEQ ID NO: 2) | |
| Human | Forward | CAGGAGAAGAAGAGGGTGGC | NM_001135.4 |
| Aggrecan | | (SEQ ID NO: 3) | |
| | Reverse | CACTGGGGTATAGGCTGGTT | |
| | | (SEQ ID NO: 4) | |
| Human type | Forward | GCCTCCCAGAACATCACCTA | NM_000088.4 |
| I collagen | | (SEQ ID NO: 5) | |
| | Reverse | TCAATCACTGTCTTGCCCCA | |
| | | (SEQ ID NO: 6) | |
| Human type | Forward | GATGCCACACTCAAGTCCCT | NM_033150.3 |
| II collagen | | (SEQ ID NO: 7) | |
| | Reverse | GTCTCGCCAGTCTCCATGTT | |
| | | (SEQ ID NO: 8) | |
| Human | Forward | ACTACAGCGAGCAGCAGCAG | NM_000346.4 |
| SOX9 | | (SEQ ID NO: 9) | |

The procedure regarding statistical analysis is as follows. All data are shown as the mean and standard deviation. Levels of significance were analyzed using two-tailed paired Student's t-test. Differences were considered statistically significant if p values were less than 0.05. P values less than 0.05 are denoted by “*”, p values less than 0.01 are denoted by “**”, and p values higher than 0.05 are denoted by “ns” (non-significant).

### Example 1

Absence of EVs Affects the Ability to Differentiate into Various Cell Lineages

To confirm the importance of EVs in cell differentiation, ADSCs were exposed to either normal FBS medium (control group) or EV-depleted FBS medium (experimental group). Cells were allowed to differentiate for 14 or 21 days and were then fixed and stained using the differentiation stain kit. The induction of adipogenic, osteogenic, and chondrogenic differentiation potential of ADSCs was observed under a light microscope.

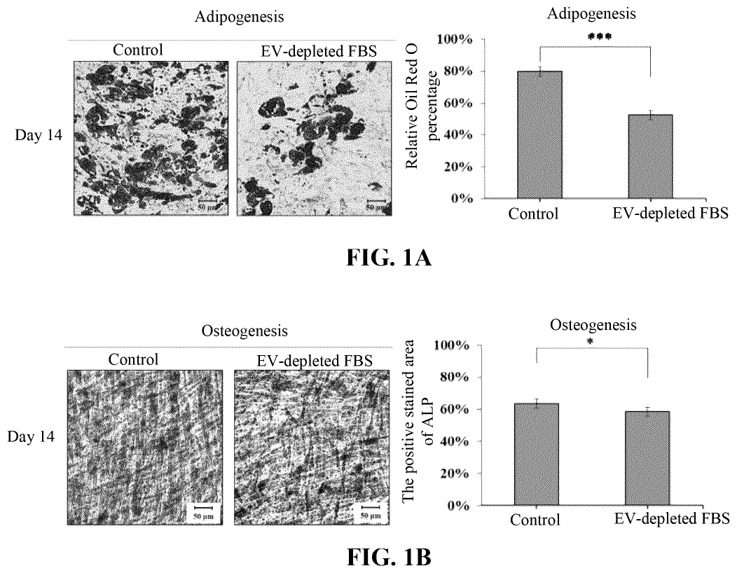

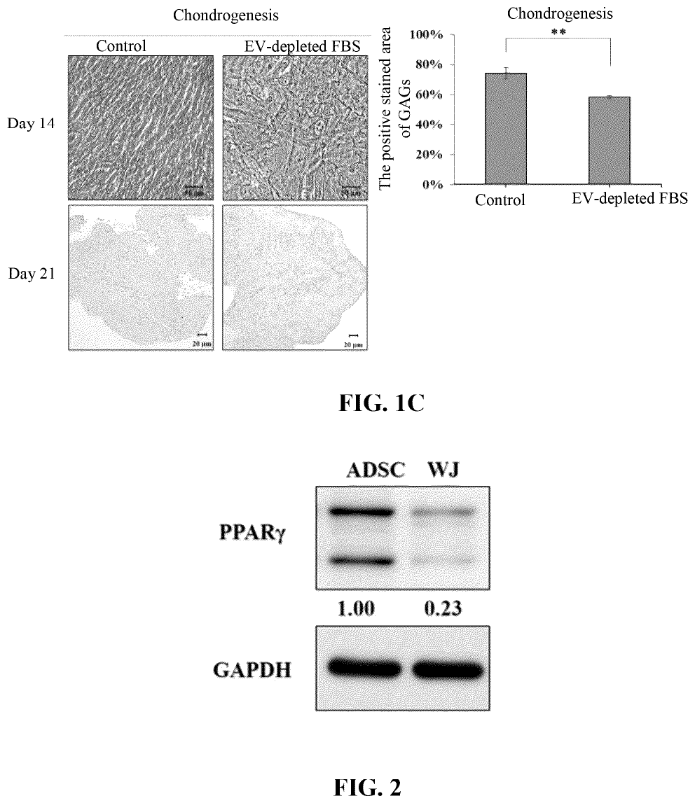

FIGS. 1A-1C show effects of adipose-derived stem cells (ADSC) differentiation on specific lineages in differentiation medium with EV-depleted FBS. ADSCs from passage 5 were cultured in various differentiation induction media with 10% FBS. (FIG. 1A) ADSCs were cultured in adipogenic differentiation medium with EV-depleted FBS for 14 days and observed by microscopy, followed by Oil Red O staining. (FIG. 1B) Cells were cultured in osteogenic differentiation medium with EV-depleted FBS for 14 days and observed by microscopy, followed by alkaline phosphatase (ALP) staining. (FIG. 1C) For chondrogenic induction, cells were maintained in differentiation medium and observed by microscopy, followed by Alcian blue staining on day 14. The histology of paraffin-embedded tissue sections was evaluated on day 21 of chondrogenic differentiation. The differentiation quantifications of tissues were performed after 14 days of adipogenic, osteogenic differentiation, and after 21 days of chondrogenic differentiation, respectively. The percentage positive area was calculated using ImageJ software. The mean±standard deviation (SD) levels of tissue differentiation in the three groups. *p<0.05; **p<0.01; ***p<0.001, when EV-depleted FBS group was compared to the control group.

The results showed that oil droplet production diminished due to treatment with EV-depleted FBS (FIG. 1A). With respect to osteogenic differentiation, the density of stained ALP in the EV-depleted group was lower than in the control group (FIG. 1B). The cartilage extracellular matrix (ECM) is composed predominantly of a collagenous network and GAGs, such as chondroitin sulfate and HA. Alcian blue is one of a group of polyvalent basic dyes that bind and precipitate the sulfate and carboxylate groups from an aqueous solution. Thus, Alcian blue can be used as a specific cartilage stain. Cartilage tissue sections were stained using Alcian blue staining, and GAG formation was reduced after EV-depleted FBS treatment (FIG. 1C). These findings indicate that the absence of EVs in the medium could affect cell differentiation, and that EVs may facilitate mesenchymal stem cell (MSC) differentiation.

### Example 2

### Secreted Substances of ADSCs Enhance Chondrocyte Differentiation Capacity

To determine whether secretory factors from ADSCs could promote ADSC differentiation into chondrocytes, we first compared the adipogenesis capacity of mesenchymal stem cells, ADSC and WJ-MSC, using the peroxisome proliferator-activated receptor gamma (PPARγ) expression level.

FIG. 2 shows adipogenic capacity of mesenchymal stem cells (MSCs) from different tissue sources. Two different tissue-derived mesenchymal stem cells, ADSCs and Wharton's jelly (WJ)-MSCs, were cultured in adipogenic differentiation medium for 14 days, and then the Western blot method was used to observe the amount of peroxisome proliferator activated receptor γ (PPARγ) protein in ADSCs and WJ-MSCs. Further ImageJ analyses of the bands were performed to measure the protein expression levels. The ratios of PPARγ protein expression was normalized to glyceraldehyde-3-phosphate dehydrogenase (GAPDH).

The results showed that the adipogenic capacity of ADSCs was superior to that of WJ-MSCs through the protein expression of PPARγ. This result showed that ADSCs have strong adipogenic differentiation ability compared with other MSCs.

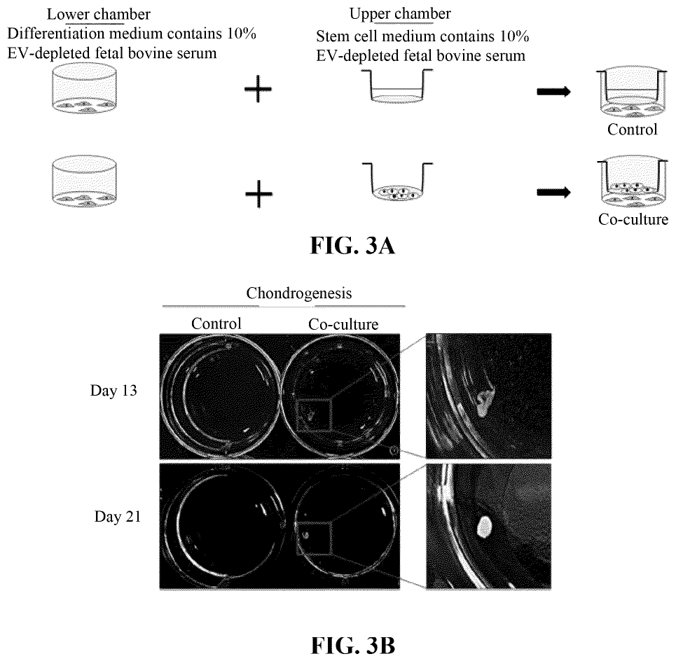

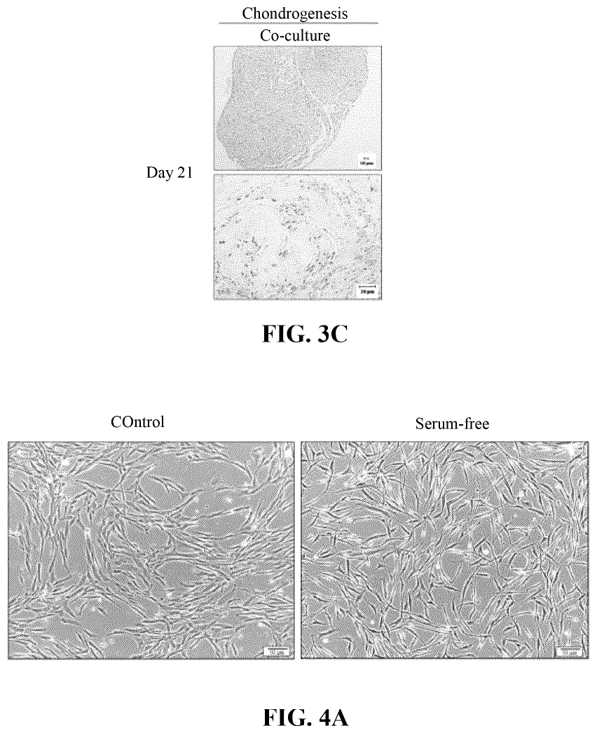

FIGS. 3A-3C show effects of chondrogenic differentiation capacity by secreted substances of ADSCs. (FIG. 3A) Scheme of the experimental procedure. ADSCs were seeded at low density (5×10³ cells/well) in the lower well, and cells at high cell densities (8×10⁴ ADSCs/well) were seeded in the upper chamber of 8.0-μm Transwell plates. The control group was the upper chamber without seeding cells. (FIG. 3B) The images recorded were used in the coculture system. Cartilage tissue was collected from the co-culture group. (FIG. 3C) The sliced cartilage tissue was stained with Alcian blue, and then cartilage differentiation status was obtained by microscopy. The nucleus was stained red-purple, and the cytoplasm was stained light pink.

Thus, we used ADSCs for further experiments. ADSCs were cultured in 10% EV-depleted FBS medium in the upper chamber using the co-culture system (FIG. 3A). The control group was not seeded with ADSCs in the upper chamber, but medium containing 10% EV-depleted FBS was also added. Then, chondrogenic differentiation-promoting factors in the upper chamber were secreted into the lower chamber. The results showed the secretion of soluble substances from ADSCs could induce the transformation of cells into chondrocyte spheroids at 13 days (FIG. 3B). GAGs are the major components of cartilage ECM and provide biological signals to stem cells and chondrocytes for cartilage development and functional regeneration. The results showed that sulfated GAG production was also detected in the ECM of cartilage tissue by histochemical assay (FIG. 3C). The cell differentiation capacity in the coculture group was better than in the control group, although the control group did not form chondrospheroids at 21 days (FIG. 3B). The results suggest that the paracrine effects of ADSCs may play a key role in the differentiation capacity of stem cells.

### Example 3

EV Production from MSCs does not Alter their Differentiation Capability

Serum-free conditions were used to induce EV production in MSCs. To assess whether the differentiation capability of ADSCs was affected by the EV collection process, cells were cultured in medium supplemented with 10% FBS for 4 days which was then replaced with SFM for 24 h. After 24 h, ADSC morphology was observed by light microscopy.

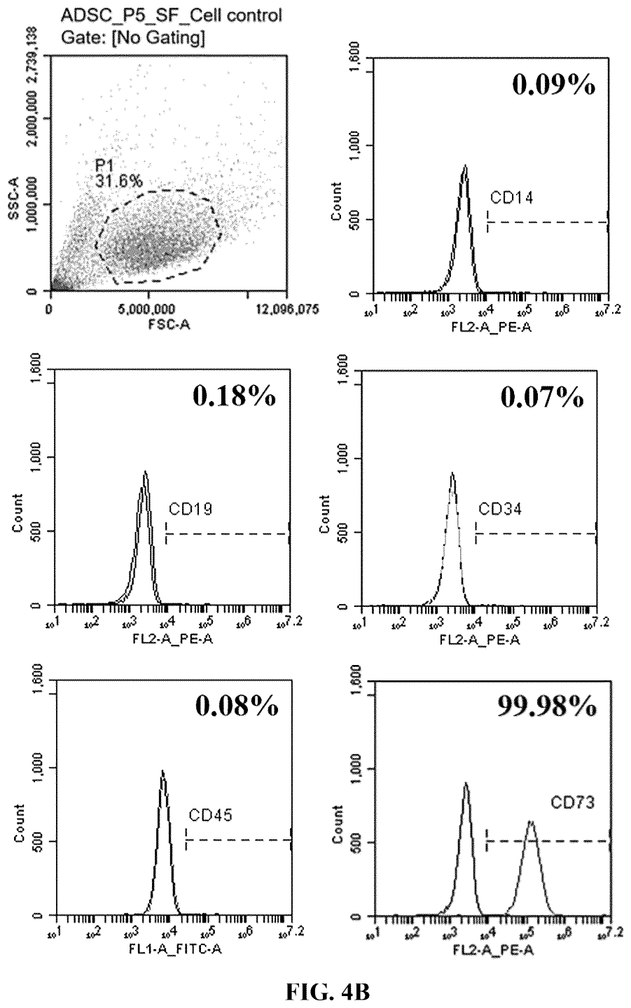

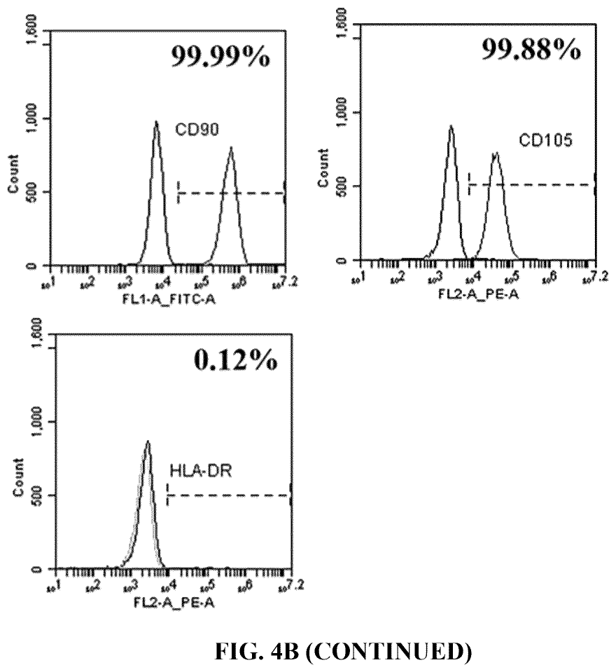

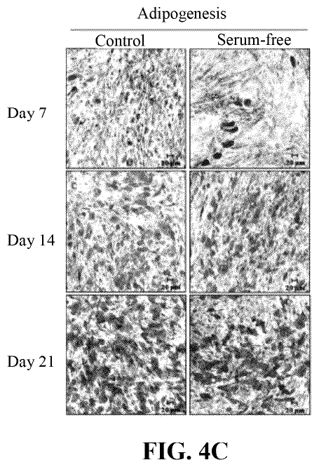

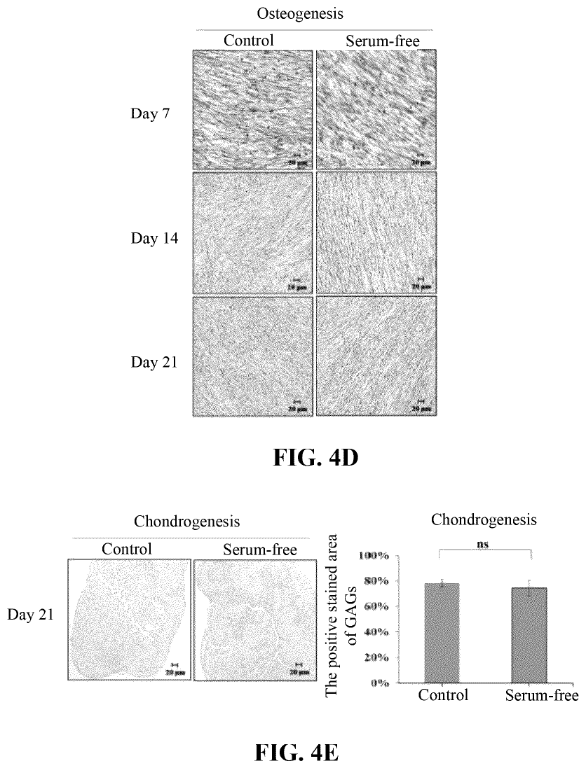

FIGS. 4A-4E show monitoring of mesenchymal stem cell (MSC) characteristics under serum-free culture conditions. The control group was cultured in medium with 10% FBS, and the serum-free group was serum starved of ADSCs for 24 h before differentiation. (FIG. 4A) ADSC morphology was observed by microscopy. Scale bar, 50 μm. (FIG. 4B) Flow cytometry was used to determine surface marker expression, including CD14, CD19, CD34, CD45, HLA-DR, CD73b, CD90, and CD105. ADSC differentiation was induced in different types of differentiation media at passage 5. (FIG. 4C) Cells that had undergone adipogenic differentiation were fixed in 4% formaldehyde, and Oil Red O staining of ADSCs for adipogenic induction was observed by microscopy at 7, 14, and 21 days. (FIG. 4D) Cells were subsequently cultured in osteogenic differentiation medium for 7, 14, and 21 days and observed by microscopy, followed by ALP staining. (FIG. 4E) For chondrogenic induction, cells were maintained in differentiation medium for 21 days. Tissue sections were stained with Alcian blue and evaluated by microscopy. The mean±standard deviation (SD) levels of chondrogenic differentiation in the three groups. A p value higher than 0.05 is denoted by “ns” (non-significant).

Spindle-shaped ADSCs exhibited morphological characteristics that were thinner and longer compared to the control group (FIG. 4A). ADSCs were further characterized by flow cytometry to examine surface markers. As shown in FIG. 4B, there was positive expression of canonical markers (CD73, CD90, and CD105) but there were no negative markers (CD14, CD19, CD34, CD45, and HLA-DR). When ADSCs were pretreated for 24 h in SFM, the differentiation capacity of ADSCs was assessed by adipogenic, osteogenic, and chondrogenic assays. The results confirmed that the ability of ADSCs to differentiate into lipid production still existed during the long-term maintenance of adipogenic differentiation medium (FIG. 4C). The osteogenic and cartilage differentiation capacities did not decrease compared to the normal FBS group (FIGS. 4D and 4E). Thus, this EV production method under serum-free conditions does not alter the characteristics of stem cells as examined by flow cytometry and stem cell differentiation assay. These results indicate that during the EV collection process, culturing under SFM condition for 24 h does not alter MSC physiological function, which suggests that under this condition, MSC-derived EV function and quality are likely not affected.

### Example 4

### EV Quantification and Characterization

Further, EVs were isolated using UF to compare the production rate of the different source cells, including ADSCs and WJ-MSCs. The particle concentration and size were measured at different passages using NTA.

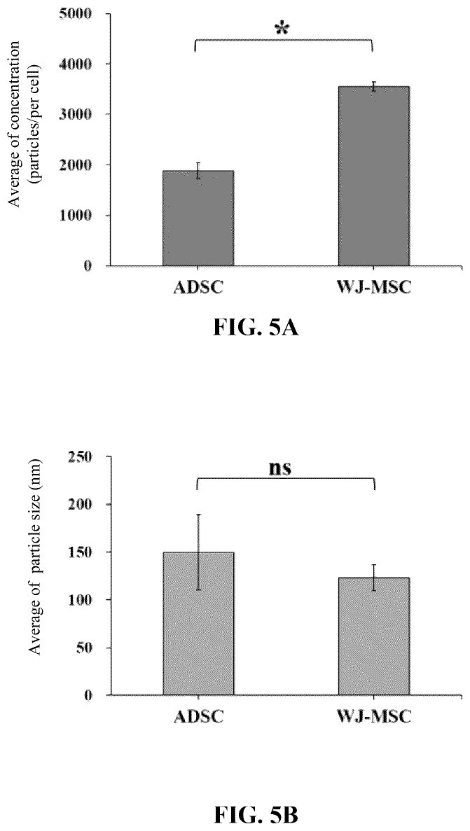

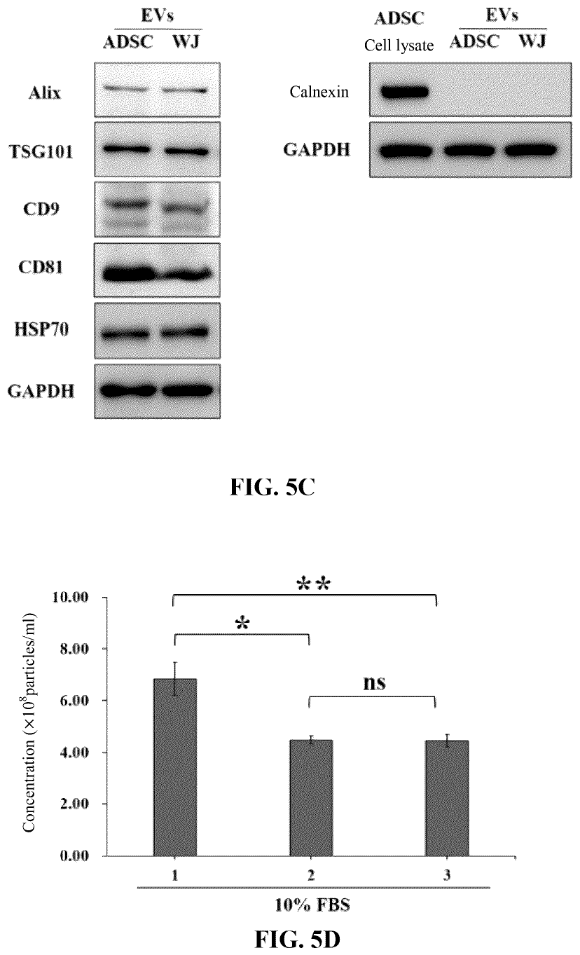

FIGS. 5A-5D show extraction and identification of MSC-derived EVs. (FIG. 5A) Using nanoparticle tracking analysis (NTA), the EV production yield from ADSCs and WJ-MSCs was analyzed by the average particle numbers in each cell. (FIG. 5B) The average size of EVs was determined by NTA. (FIG. 5C) The protein levels of EV markers, including Alix, TSG101, CD9, CD81, and HSP70, were analyzed from EV samples using Western blot analysis. Analysis of Calnexin, which is not expressed by EV, in ADSC lysates and EV samples using Western blotting. (FIG. 5D) The stock solution FBS was diluted with DPBS to a final concentration of 10% FBS. The particle concentration of EVs from 10% FBS was analyzed by NTA. This was the analysis result of the three samples. That is, the results of 3 separate samplings, each sample would be analyzed 3 times. The difference was considered statistically significant if p values were less than 0.05. P values less than 0.05 are denoted by by “*”, p values less than 0.01 are denoted by “**”, and p values higher than 0.05 are denoted by “ns” (non-significant).

The serum-free condition could produce approximately 2000 particles/per ADSC cell and secrete approximately 3500 particles/per WJ-MSC cell (FIG. 5A). The data also suggested that WJMSCs could produce more EVs than ADSCs. The average sizes of ADSC-derived EVs were 150±39 nm, whereas the average diameters of WJ-MSC-derived exosomes were 123±14 nm (FIG. 5B). EVs were also examined for the expression of the representative proteins. The results confirmed the presence of MSC-derived EV-enriched markers, such as the biogenesis-related proteins Alix (95 kDa) and TSG101 (45 kDa), HSP70 (70 kDa), CD9 (24 kDa), and CD81 (25 kDa), using Western blot analysis. Moreover, the endoplasmic reticulum protein calnexin was present in cell lysates, but absent in EVs (FIG. 5C). These data suggest most EVs may be exosomes.

In addition, FBS was diluted to 10% with sterile water, and the EV concentration was analyzed using NTA. Approximately 4.4×10⁸ to 6.8×10⁸ EVs/mL of 10% serum were obtained, indicating the EV concentration in the culture condition. Thus, about 5×10⁸ particles/mL were used to test the differentiation capacity of MSCs (FIG. 5D).

### Example 5

The EVs Maintained their Chondrogenic Differentiation Ability

Experiments were conducted to verify whether or not EVs isolated from serum-free culture medium could also promote chondrogenic differentiation capacity.

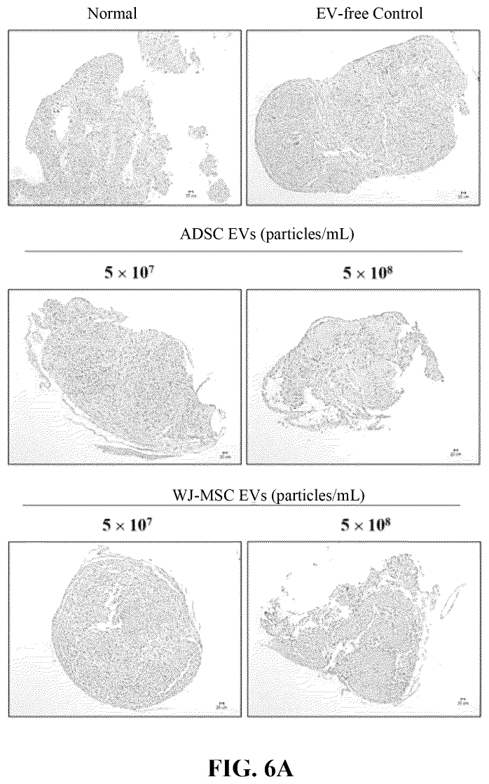

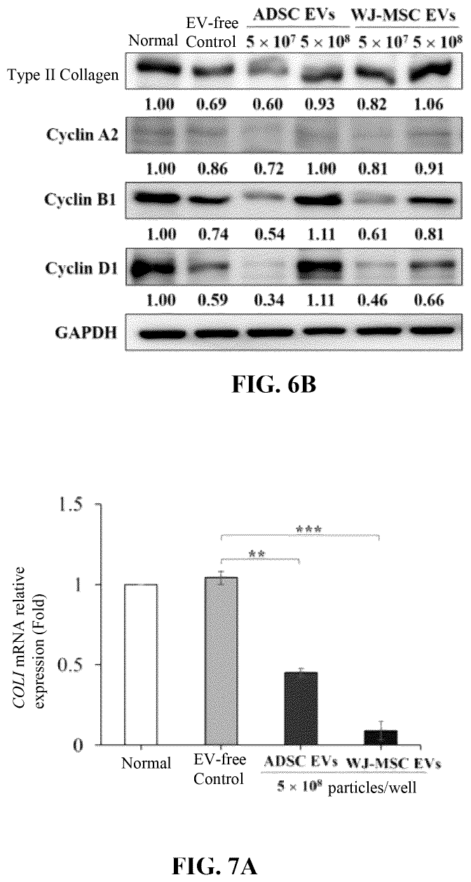

FIGS. 6A and 6B show effects of EVs on stem cell differentiation into chondrocytes. Normal FBS was added to the culture medium used for the MSC differentiation process in the regular condition group (Normal). Cells from the EV-free control group were maintained in medium with 10% EV-depleted FBS during the differentiation process. Cells from the EV test groups were cultured in a medium containing EV-deleted FBS. Cells were added with EVs derived from ADSCs and WJ-MSCs in chondrogenic differentiation medium. The EV concentrations were 5×10⁷ and 5×10⁸, respectively. The medium was completely replaced every 3 days. (FIG. 6A) Cells were cultured in chondrogenic differentiation medium for 21 days. Tissue sections were stained and observed by microscopy, followed by Alcian blue staining. (FIG. 6B) On day 14, cells were harvested, and lysis buffer was used to extract the proteins. Type II collagen and cyclins (A2, B1, and D1) protein expression levels were assessed by Western blot analysis. Further ImageJ analyses of the bands were performed to measure the protein expression levels. GAPDH represents glyceraldehyde-3-phosphate dehydrogenase.

In functional EV experiments, ADSCs were cultured in EV-free FBS medium, and cells were added with different concentrations of EVs derived from ADSCs and WJ-MSCs. At 21 days, the ability of ADSCs to differentiate into chondrocytes was observed. Moreover, treatment in EV-free conditions resulted in a decreased differentiation capacity compared to treatment in FBS-added medium, which served as the normal group. However, incubation of ADSCs at 5×10⁸ particles/mL EV concentration almost restored GSG and GP production (FIG. 6A).

At 14 days, the expression of chondrogenic differentiation and cell proliferation-related proteins in the early stage was determined. During the chondrogenic differentiation process of ADSCs, the high concentration of MSCderived EVs (5×10⁸ particles/mL) seemed to increase COLII expression compared to 5×10⁷ particles/mL. EVs enhanced the chondrogenic differentiation of ADSCs via COLII upregulation (FIG. 6B), indicating that EVs could promote early chondrogenic differentiation in vitro. Cellular proliferation requires passage through a cell cycle to produce new cells. The cell proliferation status and the expression of the cell cycle-related proteins cyclins A, B, and D were examined. During differentiation induction, the results showed cyclin A2 and cyclin D1 upregulation after EV treatment, which contributed to the stimulation of DNA synthesis. Treatment of ADSCs with EVs also triggered cyclin B protein expression (FIG. 6B). That is to say, cells retained their ability to divide with the addition of EVs, thereby allowing growth during cell differentiation.

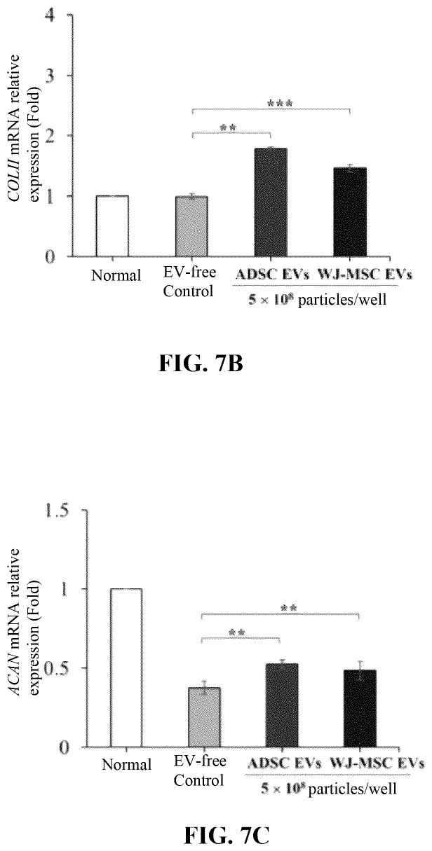

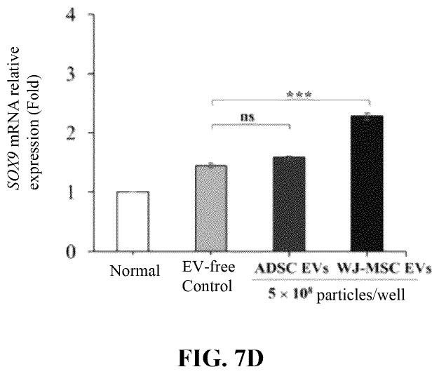

In addition, we analyzed whether treatment with MSC-derived EV altered mRNA expression of collagen type I (COLI), COLII, aggrecan (ACAN), and SOX9 during chondrogenic differentiation (FIGS. 7A-7D).

FIGS. 7A-7D show quantitative real-time gene expression analysis of chondrogenic-specific genes. Normal FBS was added to the culture medium used for the MSC differentiation process in the regular condition group (Normal). Cells from the EV-free control group were maintained in medium with 10% EV-depleted FBS during the differentiation process. Cells from the EV test groups were cultured in a medium containing EV-deleted FBS. Cells were added with EVs derived from ADSCs and WJ-MSCs in chondrogenic differentiation medium. The mRNA expression levels of (FIG. 7A) type I collagen (COLI), (FIG. 7B) COLII, (FIG. 7C) aggrecan (ACAN), and (FIG. 7D) SOX9, were measured in chondrogenic differentiated tissues after 21 days. GAPDH was used as an internal control. The value of fold change was normalized to the regular culture condition, and all values are expressed as mean±SEM, n=3. *p<0.05; **p<0.01, compared to the EV-free group using t-test. A p value higher than 0.05 is denoted by “ns” (non significant).

The results showed that under treatment with either ADSC-derived EVs or WJMSC-derived EVs, both types could reduce COLI μmRNA expression levels, while enhancing mRNA expression levels of COLII, ACAN, and SOX9 compared to the EVfree condition. These data suggest that EVs could upregulate the expression levels of chondrogenesis-related genes to promote chondrogenic differentiation.

### Example 6

### MSC-Derived EVs Reduce Lipid Droplet Formation

Experiments were performed to examine whether or not EVs secreted from the cell culture medium affect fat production. In the differentiation process, the effects of the adipogenic ability of both EVs from FBS and the addition of EVs derived from different MSCs were compared.

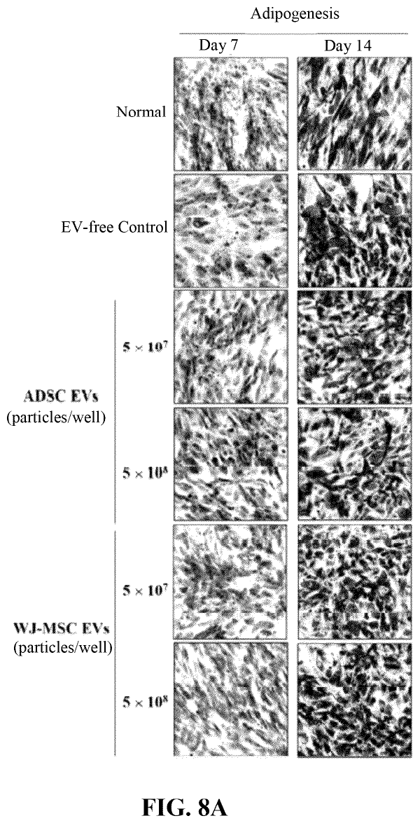

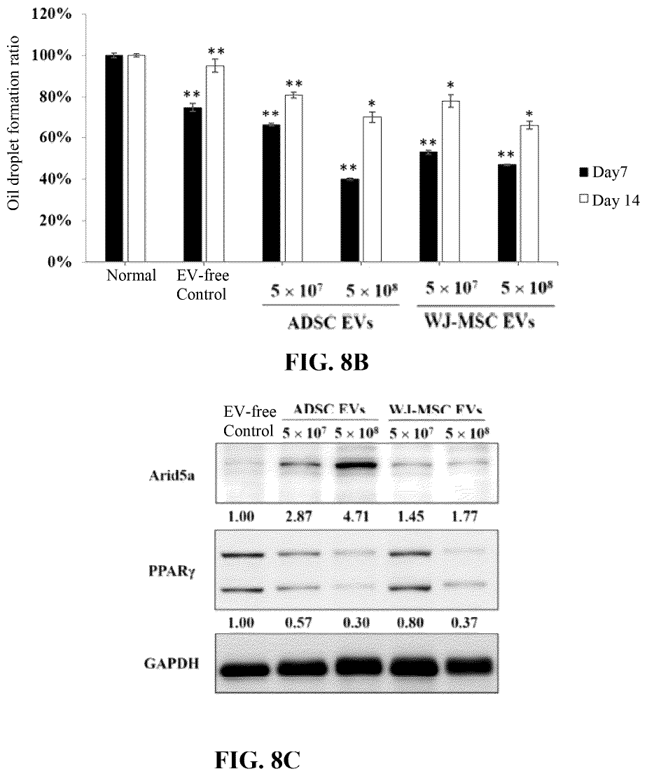

FIGS. 8A-8C show effects of EVs on stem cell differentiation into adipocytes. During the differentiation process, EVs derived from ADSCs and WJ-MSCs were added to adipogenic differentiation medium and treated with different EV concentrations. The medium was replaced every 3 days. (FIG. 8A) On days 7 and 14, lipid accumulation during adipogenic induction was monitored by Oil Red O staining. Hematoxylin-stained cell nuclei were stained purplish blue. (FIG. 8B) Lipid droplets were dissolved in methanol, and the amount of oil droplet generation was quantified. (FIG. 8C) On day 14, cells were harvested, and lysis buffer was used to extract the proteins. AT-rich interactive domain 5A (Arid5a) and PPARγ protein expression levels were assessed by Western blot analysis. Further ImageJ analyses of the bands were performed to measure the protein expression levels. The ratios of Arid5a and PPARγ protein expression were normalized to GAPDH. *p<0.05; **p<0.01, compared to the normal group using t-test.

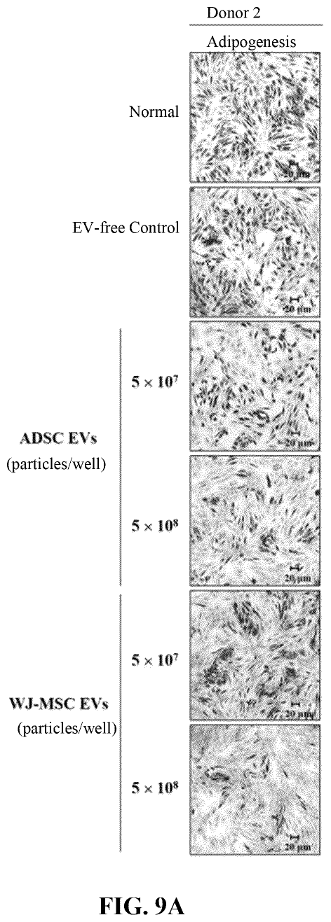

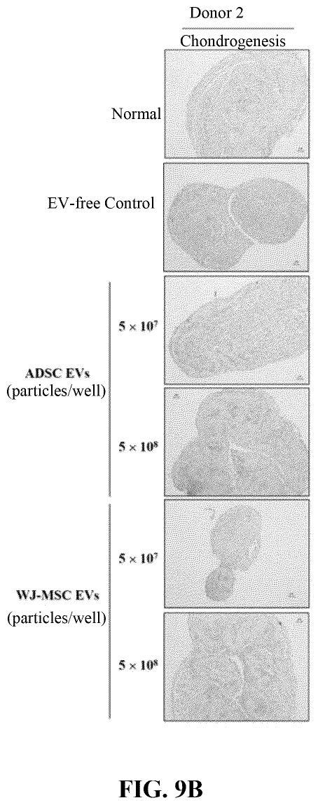

The staining results showed that MSC-derived EVs could significantly mitigate the volume of the oil droplets (FIG. 8A). The quantitative results also indicated that EV extracted from the stem cell culture medium could alleviate lipid accumulation (FIG. 8B). Furthermore, we explored the expression of adipogenesis-related proteins, including AT-rich interactive domain 5a (Arid5a) and PPARγ. Arid5a is a negative regulator during adipogenic differentiation and it inhibits adipogenesis by inhibiting the transcription of PPARγ. The results showed that treatment with MSC-derived EVs significantly reduced the expression levels of PPARγ, and the protein expression levels of Arid5a were upregulated (FIG. 8C). Thus, these results confirmed that both ADSC- and WJ-MSC-derived EVs decreased the adipogenic induction ability, but both ADSC- and WJMSC-derived EVs did not completely inhibit lipid production. We also used ADSCs from another donor as target cells for the chondrogenic and adipogenic differentiation assay to avoid donor effects (FIGS. 9A and 9B).

FIGS. 9A and 9B show the second donor's adipogenic differentiation and chondrogenic differentiation experiments. (FIG. 9A) On days 14, lipid accumulation during adipogenic induction was monitored by Oil Red O staining. Hematoxylin-stained cell nuclei were stained purplish blue. (FIG. 9B) Cells were cultured in chondrogenic differentiation medium for 21 days. Tissue sections were stained and observed by microscopy, followed by Alcian blue staining.

The data indicate that both ADSC-derived EV and WJ-MSC derived EV could not only successfully promote chondrogenic differentiation, but could also reduce lipid production in the second donor's ADSCs.

In summary, both ADSC-derived EVs and WJMSC-derived EVs could not only upregulate chondrogenesis-related protein expression levels, but could also downregulate adipogenesis-related protein expression levels, which could result in promotion of MSC differentiation to chondrocytes.

### Example 7

### Analysis of Cytokine and Growth Factor Content in MSC-Derived EVs

Mesenchymal stem cells secrete biologically active molecules such as a variety of growth factors, proteins, cytokines, signaling lipids, mRNA and microRNA (miRNA), and carry them to target cells via extracellular vesicles (EVs). EVs are important mediators of intercellular communication and participate in regulating physiological processes.

The EVs produced as mentioned above were analyzed with MILLIPLEX® MAP MULIPLEX DETECTION (Merck Milliplex, instrument model: Luminex Magpix analyzer) for analysis contents of interleukin-2 (IL-2), Uteroglobin, alpha-fetoprotein (AFP), angiopoietin like 6 (ANGPTL-6), fatty acid-binding protein-1 (FABP-1), cartilage oligomeric matrix protein (COMP), platelet-derived growth factor AA (PDGF-AA), and granulocyte-colony stimulating factor (G-CSF). The values are recorded in Table 2.

TABLE 2

| Factor (pg/ml) | Control | ADSC-EVs | WJ-EVs |
|---|---|---|---|
| IL-2 | 0.27 | 0.28 | 0.24 |
| Uteroglobin | 1.92 | 2.08 | 2.25 |
| AFP | 0.01 | 0.01 | 0.01 |
| ANGPTL-6/AGF | 0.45 | 0.44 | 0.42 |
| FABP-1 | 0.02 | 0.02 | 0.02 |
| COMP | 2.79 | 128.57 | 9.26 |
| PDGF-AA | 5.12 | 78.26 | 219.45 |
| G-CSF | 4.03 | 1465.00 | 542.58 |

As shown in Table 2, the mesenchymal stem cell (MSC)-derived extracellular vesicle (EV) of the present invention regulates the expression levels of IL-2, Uteroglobin, AFP, ANGPTL-6, FABP-1, COMP and PDGF-AA. These data indicate that EVs can increase the expression levels of chondrogenesis-related genes to promote chondrogenesis.

As shown in Table 2, the mesenchymal stem cell (MSC)-derived extracellular vesicle (EV) of the present invention significantly increases the expression level of G-CSF. The data suggests that EVs can upregulate the expression levels of anti-inflammatory genes to inhibit adipogenesis.

In summary, the mesenchymal stem cell (MSC)-derived extracellular vesicle (EV) of the present invention achieves the effect on reducing adipogenesis and treating osteoarthritis through upregulating type II collagen, decreaseing lipid accumulation rate during adipogenic induction, and reducing lipid droplet formation.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for preparing a mesenchymal stem cell (MSC)-derived extracellular vesicle (EV), comprising the following steps:
(a) providing a human mesenchymal stem cell;
(b) culturing and maintaining the human mesenchymal stem cell in a keratinocyte serum-free medium (KSFM) supplemented with fetal bovine serum (FBS), N-acetyl-L-cysteine and L-ascorbic acid 2-phosphate;
(c) collecting a cultured human mesenchymal stem cell, and removing cell debris using a centrifugation process; and
(d) filtering supernatant thus formed to obtain the mesenchymal stem cell (MSC)-derived extracellular vesicle (EV).

2. The method according to claim 1, wherein the human mesenchymal stem cell is adipose-derived mesenchymal stem cell (ADSC).

3. The method according to claim 1, wherein the mesenchymal stem cell-derived extracellular vesicle has a size of 30 nm-1 μm.

4. A mesenchymal stem cell (MSC)-derived extracellular vesicle (EV), which is prepared by the method according to claim 1.

5. The mesenchymal stem cell (MSC)-derived extracellular vesicle (EV) according to claim 4, wherein the human mesenchymal stem cell is adipose-derived mesenchymal stem cell (ADSC).

6. The mesenchymal stem cell (MSC)-derived extracellular vesicle (EV) according to claim 4, having a size of 30 nm-1 μm.

7. The mesenchymal stem cell (MSC)-derived extracellular vesicle (EV) according to claim 4, comprising a cartilage-related gene selected from the group consisting of: interleukin-2 (IL-2), Uteroglobin, alpha-fetoprotein (AFP), angiopoietin-like 6 (ANGPTL-6), fatty acid-binding protein-1 (FABP-1), cartilage oligomeric matrix protein (COMP), platelet-derived growth factor AA (PDGF-AA), granulocyte-colony stimulating factor (G-CSF), and a combination thereof.

8. A method for reducing adipogenesis, comprising administering to a subject in need thereof a pharmaceutical composition comprising an effective amount of the mesenchymal stem cell (MSC)-derived extracellular vesicle (EV) according to claim 4.

9. The method according to claim 8, wherein the mesenchymal stem cell (MSC)-derived extracellular vesicle (EV) is adipose-derived mesenchymal stem cell (ADSC)-derived extracellular vesicle (EV).

10. The method according to claim 8, wherein the mesenchymal stem cell-derived extracellular vesicle has a size of 30 nm-1 μm.

11. The method according to claim 8, wherein the mesenchymal stem cell-derived extracellular vesicle upregulates type II collagen.

12. The method according to claim 8, wherein the mesenchymal stem cell-derived extracellular vesicle decreases lipid accumulation rate during adipogenic induction.

13. The method according to claim 8, wherein the mesenchymal stem cell-derived extracellular vesicle reduces lipid droplet formation.

14. The method according to claim 8, wherein the pharmaceutical composition is in a dosage form for parenteral administration.

15. A method for treating osteoarthritis (OA), comprising administering to a subject in need thereof a pharmaceutical composition comprising an effective amount of the mesenchymal stem cell (MSC)-derived extracellular vesicle (EV) according to claim 4.

16. The method according to claim 15, wherein the mesenchymal stem cell (MSC)-derived extracellular vesicle (EV) is adipose-derived mesenchymal stem cell (ADSC)-derived extracellular vesicle (EV).

17. The method according to claim 15, wherein the mesenchymal stem cell-derived extracellular vesicle has a size of 30 nm-1 μm.

18. The method according to claim 15, wherein the mesenchymal stem cell-derived extracellular vesicle produces chondrocyte.

19. The method according to claim 15, wherein the osteoarthritis is caused by obesity.

20. The method according to claim 15, wherein the pharmaceutical composition is in a dosage form for parenteral administration.


SEQUENCE LISTING (9 sequences)

SEQ ID NO: 1 - DNA, 20 bp, synthetic construct
  source 1..20: mol_type = other DNA
gtcaaggctgagaacgggaa

SEQ ID NO: 2 - DNA, 20 bp, synthetic construct
  source 1..20: mol_type = other DNA
gcagagggggcagagatgat

SEQ ID NO: 3 - DNA, 20 bp, synthetic construct
  source 1..20: mol_type = other DNA
caggagaagaagagggtggc

SEQ ID NO: 4 - DNA, 20 bp, synthetic construct
  source 1..20: mol_type = other DNA
cactggggtataggctggtt

SEQ ID NO: 5 - DNA, 20 bp, synthetic construct
  source 1..20: mol_type = other DNA
gcctcccagaacatcaccta

SEQ ID NO: 6 - DNA, 20 bp, synthetic construct
  source 1..20: mol_type = other DNA
tcaatcactgtcttgcccca

SEQ ID NO: 7 - DNA, 20 bp, synthetic construct
  source 1..20: mol_type = other DNA
gatgccacactcaagtccct

SEQ ID NO: 8 - DNA, 20 bp, synthetic construct
  source 1..20: mol_type = other DNA
gtctcgccagtctccatgtt

SEQ ID NO: 9 - DNA, 20 bp, synthetic construct
  source 1..20: mol_type = other DNA
actacagcgagcagcagcag